(19) United States
(12) Patent Application Publication
(10) Pub. No.: US 2022/0030199 A1
(43) Pub. Date: Jan. 27, 2022

(54) ENDOSCOPE SYSTEM, INFORMATION PROVISION METHOD, AND RECEIVER

(71) Applicant: OLYMPUS CORPORATION, Tokyo (JP)

(72) Inventors: Takashi NAGATA, Tokyo (JP); Kazuhiro Akiyama, Tokyo (JP); Isao TATESHITA, Tokyo (JP); Kei MITSUHASHI, Tokyo (JP)

(73) Assignee: OLYMPUS CORPORATION, Tokyo (JP)

(21) Appl. No.: 17/496,316

(22) Filed: Oct. 7, 2021

Related U.S. Application Data
(63) Continuation of application No. PCT/JP2019/016016, filed on Apr. 12, 2019.

(51) Int. Cl. H04N 7/18; G16H 40/63; G16H 30/20

(52) U.S. Cl. CPC H04N 7/181; G16H 30/20; G16H 40/63

(57) ABSTRACT

A connection detection unit detects that a receiver is connected that is capable of recording an image data group captured by a capsule endoscope. A provision unit provides a plurality of pieces of subject information to a plurality of receivers detected to be connected.

### CROSS-REFERENCE TO RELATED APPLICATIONS

This application is based upon and claims the benefit of priority from International Application No. PCT/JP2019/016016, filed on Apr. 12, 2019, the entire contents of which are incorporated herein by reference.

### BACKGROUND

### 1. Field of the Disclosure

The present disclosure relates to a technology for providing subject information to a receiver of image data.

### 2. Description of the Related Art

Japanese Patent Application Publication No. 2009-61123 discloses a system including: a receiver for receiving an image signal wirelessly transmitted by a capsule endoscope; and a workstation. In the system described in Japanese Patent Application Publication No. 2009-61123, the receiver is connected to the workstation and has examination information and subject information transmitted from the workstation before the start of an examination. The receiver is then removed from the workstation and attached to the subject and performs an examination that records approximately eight hours of internal body images transmitted from a capsule endoscope that has been swallowed. After the examination by the capsule endoscope is completed, the receiver is removed from the subject and then connected to the workstation again so as to transfer the received internal body images to the workstation in association with the examination ID.

In a conventional capsule endoscope system, a nurse operates a workstation to transmit examination information and subject information to a receiver before an examination. If multiple capsule endoscopic examinations are scheduled for one day, the nurse transmits examination information and subject information to be assigned to each of multiple receivers from the workstation, and each receiver records the examination information and the subject information that are assigned to the receiver. After that, when attaching a receiver to the subject, the nurse has the examination information and the subject information recorded in the receiver to be displayed for checking in order to prevent mixing up. The nurse has to transmit and check examination information and subject information as many times as the number of receivers used for the examination as described, which is time-consuming.

### SUMMARY

In this background, a purpose of the present disclosure is to provide a technology for efficiently providing subject information to a receiver.

An examination management apparatus according to one embodiment of the present disclosure includes: a connection detection unit that detects that a receiver is connected that is capable of recording an image data group captured by a capsule endoscope; and a provision unit that provides the same subject information at a predetermined time to all of a plurality of receivers detected to be connected.

Optional combinations of the aforementioned constituting elements and implementations of the disclosure in the form of methods, apparatuses, systems, recording mediums, and computer programs may also be practiced as additional modes of the present disclosure.

### BRIEF DESCRIPTION OF THE DRAWINGS

Embodiments will now be described, by way of example only, with reference to the accompanying drawings that are meant to be exemplary, not limiting, and wherein like elements are numbered alike in several figures, in which:

FIG. 1 is a diagram for explaining the outline of a capsule endoscope system according to an embodiment;

FIG. 2 is a diagram showing functional blocks of an examination management apparatus;

FIG. 3 is a diagram showing an example of a status management table for examination orders;

FIG. 4 is a diagram showing recorded contents of the status management table after a change;

FIG. 5 is a diagram showing a state in which another receiver is connected to the examination management apparatus;

FIG. 6 is a diagram showing a state in which another receiver is connected to the examination management apparatus; and

FIG. 7 is a diagram showing recorded contents of the status management table after a change.

### DETAILED DESCRIPTION

The disclosure will now be described by reference to the preferred embodiments. This does not intend to limit the scope of the present disclosure, but to exemplify the disclosure.

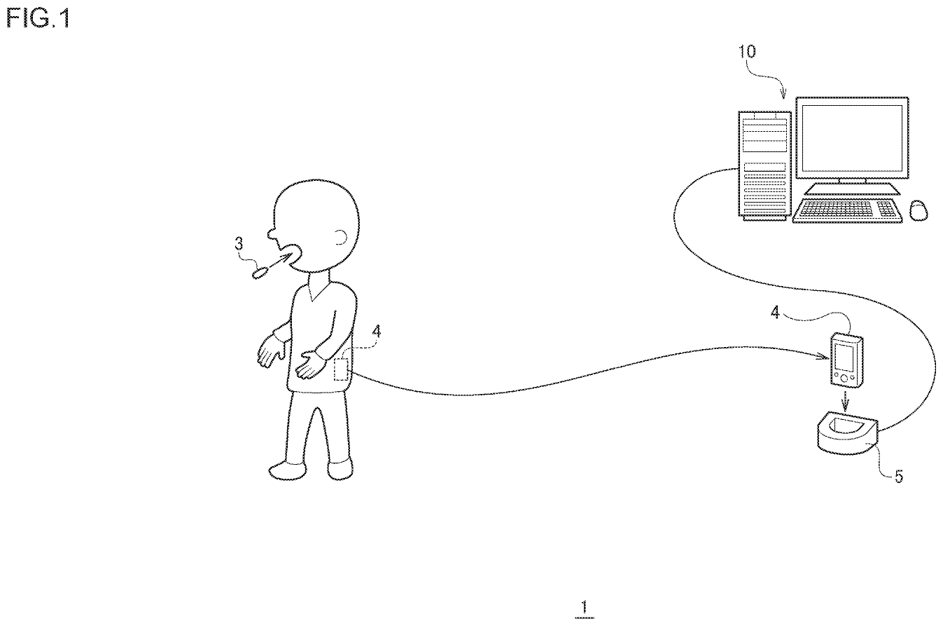

FIG. 1 is a diagram for explaining the outline of a capsule endoscope system according to an embodiment. A capsule endoscope system 1 includes a receiver 4 capable of recording an image data group captured by a capsule endoscope 3, an examination management apparatus 10 that manages capsule endoscopic examinations, and a cradle 5 that is connected to the examination management apparatus 10 in a data transferable manner. The cradle 5 is connected to the examination management apparatus 10 by a data transfer cable. The receiver 4 includes a display for displaying information and an input unit for operating displayed items. The input unit may be a button or the like, and the display may be configured as a touch panel. The receiver 4 is charged by being inserted into the cradle 5 and is connected to the examination management apparatus 10 via the cradle 5.

In a capsule endoscopic examination, after having a plurality of receiver antennas (not shown) attached to the abdomen and a receiver 4 attached to the waist by a belt, the subject (patient) swallows a capsule endoscope 3 from the mouth. The capsule endoscope 3 includes: an image capturing unit, which is an ultra-small camera; an illumination unit that illuminates the inside of the body of the subject; a signal processing unit that performs an A/D conversion of an image-capturing signal that is output from the image capturing unit and generates image data to which an image ID and image-capturing time information are added; a memory that temporarily stores image data; a communication module that transmits image data stored in the memory; and a battery that supplies power to each unit. The image ID is information for identifying the image and may be information to which a serial number indicating the image-capturing order is assigned. The capsule endoscope 3 captures still images periodically as the capsule endoscope 3 moves through the digestive tract and transmits image data to the receiver 4 through the antennas.

A recording medium is built in the receiver 4, and the receiver 4 records image data received by each receiver antenna in the recording medium. In the case where the capsule endoscope 3 images the inside of the body every 0.5 seconds, about 60,000 pieces of image data are recorded in the recording medium when the imaging of the inside of the body is completed in about eight hours.

When the imaging of the inside of the body is completed, the antennas and the receiver 4 are collected from the subject. When a nurse inserts the receiver 4 into the cradle 5, a data terminal of the receiver 4 is connected to a connector of the cradle 5, and about 60,000 pieces of image data recorded in the recording medium are transferred to the examination management apparatus 10.

The examination management apparatus 10 is realized by a workstation, manages examination orders, and records image data captured by the capsule endoscope 3.

Before the start of an examination, one examination order is assigned to the receiver 4, and after the examination is completed, the receiver 4 transfers image data to the examination management apparatus 10 in association with information for identifying the examination order. Thereby, the examination management apparatus 10 records the image data captured in the capsule endoscopic examination in association with information (examination ID) for identifying the examination. The information for identifying the examination order is typically the examination ID. Alternatively, the information may be information for identifying the subject (subject ID).

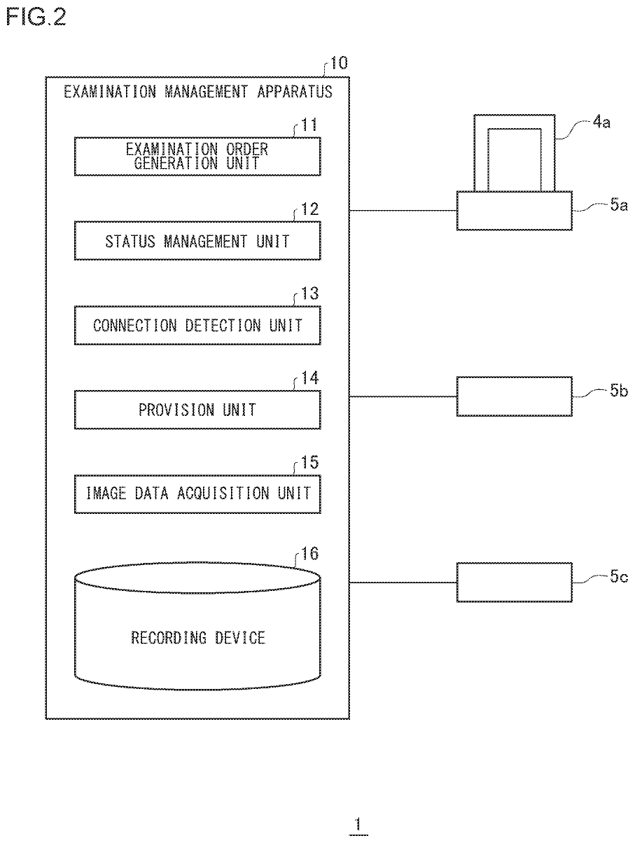

FIG. 2 shows functional blocks of the examination management apparatus 10. The examination management apparatus 10 is connected to a plurality of cradles 5 and includes an examination order generation unit 11, a status management unit 12, a connection detection unit 13, a provision unit 14, an image data acquisition unit 15, and a recording device 16. In the example shown in FIG. 2, three cradles 5a, 5b, and 5c are connected to the examination management apparatus 10. However, the number of connected cradles 5 is not limited to three.

The configuration of the examination management apparatus 10 is implemented by hardware such as a processor, a memory, or other LSIs and by software such as a program or the like loaded into the memory. The figure depicts functional blocks implemented by the cooperation of hardware and software. Thus, a person skilled in the art should appreciate that there are many ways of accomplishing these functional blocks in various forms in accordance with the components of hardware only, software only, or the combination of both.

The examination order generation unit 11 generates an examination order for a capsule endoscopic examination. A doctor may enter necessary information into a predetermined input format, and the examination order generation unit 11 may thereby generate an examination order. The information of the examination order (order information) includes at least the examination ID and information related to the subject (subject information). The subject information may include subject ID, name, sex, age, contraindication information, and the like. The order information may further include examination date. In the capsule endoscope system 1, the examination order may be generated by a hospital information system (HIS) and provided to the examination management apparatus 10.

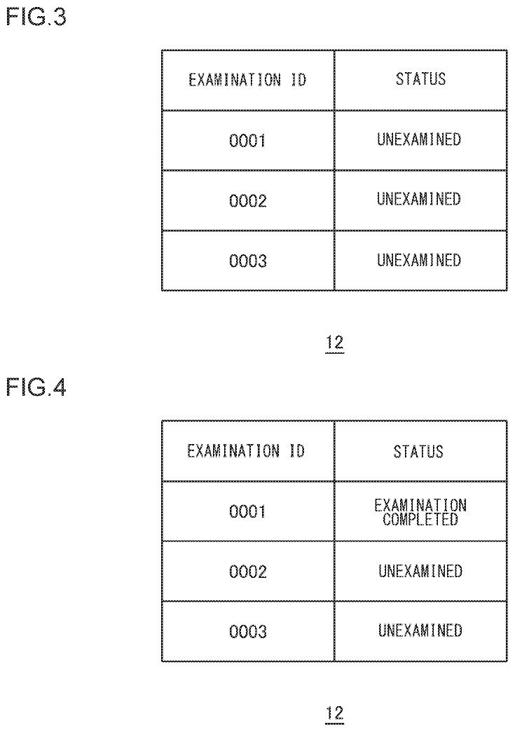

The status management unit 12 manages the status of an examination order. FIG. 3 shows an example of a status management table for examination orders. The status management table records relationships between examination IDs and statuses. The status management unit 12 manages the respective statuses of a plurality of examination orders in the status management table. When an examination order is generated by the examination order generation unit 11, the status management unit 12 newly registers the examination ID of the examination order in the status management table and sets the status of the examination order to “unexamined”. The “unexamined” status indicates that an image data group has not yet been recorded in the recording device 16.

When the capsule endoscopic examination is completed, the receiver 4 that has recorded image data is removed from the subject and inserted into a cradle 5. The connection detection unit 13 constantly monitors the connection state of the cradle 5 and detects that the receiver 4 is connected when the receiver 4 is inserted into the cradle 5 and the terminal of the receiver 4 comes into contact with the connector of the cradle 5.

At this time, the image data acquisition unit 15 makes an inquiry to the receiver 4 to see whether an image data group recorded during the capsule endoscopic examination exists. If an image data group exists, the image data acquisition unit 15 acquires the image data group along with information for identifying the examination order assigned to the receiver 4 and records the image data group in the recording device 16. When the acquisition of the image data group by the image data acquisition unit 15 is completed, the status management unit 12 changes the status of the examination order from “unexamined” to “examination completed”. The “examination completed” status indicates that the image data group has been recorded in the recording device 16.

Before the start of the examination, one examination order is assigned to the receiver 4. Conventionally, a nurse operates an examination management apparatus and manually assigns one examination order to one receiver 4. However, in the embodiment, the examination management apparatus 10 automatically provides the receiver 4 information regarding all examination orders with the “unexamined” status. Therefore, if there are a plurality of “unexamined” examination orders, information regarding the plurality of examination orders is provided to the receiver 4. When attaching the receiver 4 to the subject, a nurse displays information regarding a plurality of examination orders on the display of the receiver 4 and selects a piece of information regarding an examination order to be performed. The information displayed on the display includes subject information. The nurse selects a piece of information indicating a subject who wears the receiver 4 from a plurality of pieces of subject information included in a subject selection screen and then assigns one examination order to the receiver 4. As described, since the nurse assigns an examination order to the receiver 4 when attaching the receiver 4 to the subject, mixing up of the receivers 4 does not occur.

The provision unit 14 provides information regarding an “unexamined” examination order to the receiver 4 that has been detected to be connected by the connection detection unit 13. The information regarding the examination order includes at least the subject information to be displayed on the subject selection screen. Further, the information regarding the examination order preferably includes the examination ID. The provision unit 14 provides the information regarding the “unexamined” examination order to the receiver 4 at a predetermined time.

It is assumed that the receiver 4a shown in FIG. 2 is assigned an examination order with an examination ID “0001” and is in a state where an examination has already been performed. At this time, the image data group captured by the capsule endoscope 3 is recorded in the receiver 4a. When the receiver 4a is inserted into the cradle 5a, the connection detection unit 13 detects that the receiver 4a is connected, and the image data acquisition unit 15 makes an inquiry to the receiver 4a to see whether a recorded image data group exists. Upon confirming that an image data group exists in the receiver 4a, the image data acquisition unit 15 transmits a transfer request to the receiver 4a so as to acquire the image data group.

When the acquisition of the image data group by the image data acquisition unit 15 is completed, the status management unit 12 changes the status of the examination order from “unexamined” to “examination completed”. FIG. 4 shows recorded contents of the status management table after the change. The status management unit 12 changes the status of the examination order with the examination ID “0001” from “unexamined” to “examination completed” and then registers the status. When the image data group is transferred from the receiver 4a and the status of the examination order is changed, the provision unit 14 provides information regarding examination orders to which the “unexamined” status is set to all receivers 4 that are detected to be connected. Since only one receiver 4a is connected in this case, the provision unit 14 provides information regarding examination orders with an examination ID “0002” and an examination ID “0003” to which the “unexamined” status is set to the receiver 4a. Therefore, the information regarding the examination orders with the examination IDs “0002” and “0003” is registered in the receiver 4a. As described, when the status management table is changed, the provision unit 14 may provide a connected receiver 4 with information regarding examination orders to which the “unexamined” status is set. Since the provision unit 14 automatically provides the information regarding the examination orders to the receiver 4, the nurse does not have to operate the examination management apparatus 10 to manually transmit the information regarding the examination orders to the receiver 4.

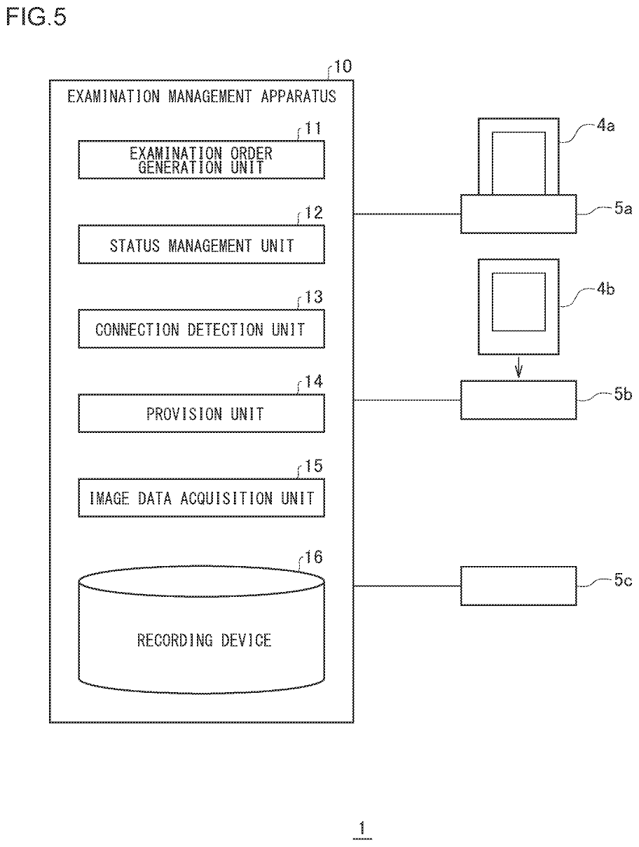

FIG. 5 shows a state in which another receiver 4b is connected to the examination management apparatus 10. No image data group is recorded in the receiver 4b. When the receiver 4b is inserted into the cradle 5b, the connection detection unit 13 detects that the receiver 4b has become newly connected, and the image data acquisition unit 15 makes an inquiry to the receiver 4b to see whether a recorded image data group exists. Upon confirming that an image data group does not exist in the receiver 4b, the image data acquisition unit 15 recognizes that it is not necessary to transfer image data from the receiver 4b.

When it is confirmed that the receiver 4b is newly connected to the examination management apparatus 10 and it is not necessary to transfer image data, the provision unit 14 may provide information regarding examination orders to which the “unexamined” status is set to all the plurality of receivers 4a and 4b that are detected to be connected. That is, the provision unit 14 provides information regarding the same plurality of examination orders to the plurality of receivers 4a and 4b at the same time. Information regarding examination orders provided in the past is registered in the receivers 4a and 4b. However, this information is overwritten by the newly provided information regarding examination orders. Thereby, the information regarding the examination orders with the examination IDs “0002” and “0003” is registered in the receivers 4a and 4b.

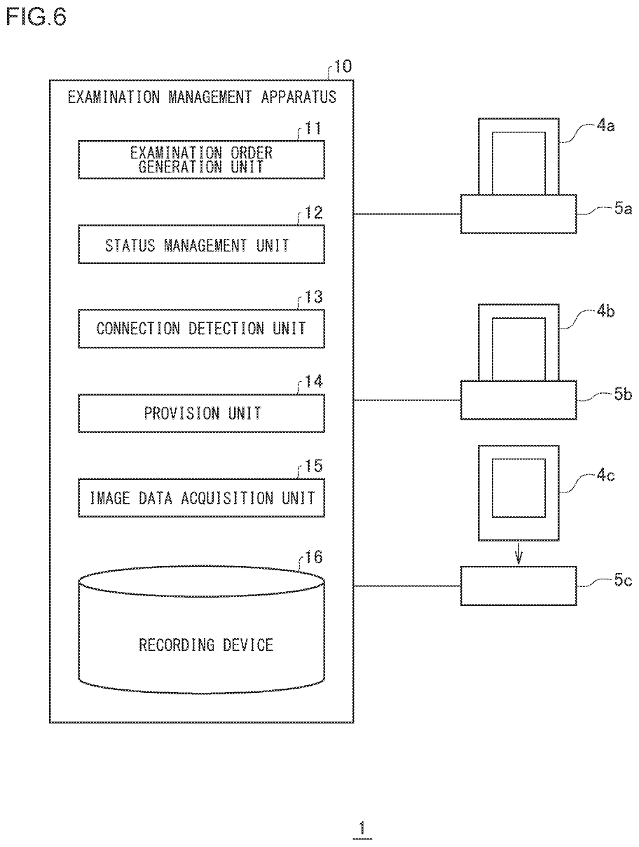

FIG. 6 shows a state in which another receiver 4c is connected to the examination management apparatus 10. It is assumed that the receiver 4c is assigned an examination order with the examination ID “0003” and is in a state where an examination has already been performed. At this time, the image data group captured by the capsule endoscope 3 is recorded in the receiver 4c. When the receiver 4c is inserted into the cradle 5c, the connection detection unit 13 detects that the receiver 4c has become newly connected, and the image data acquisition unit 15 makes an inquiry to the receiver 4c to see whether a recorded image data group exists. Upon confirming that an image data group exists in the receiver 4c, the image data acquisition unit 15 transmits a transfer request to the receiver 4c so as to acquire the image data group.

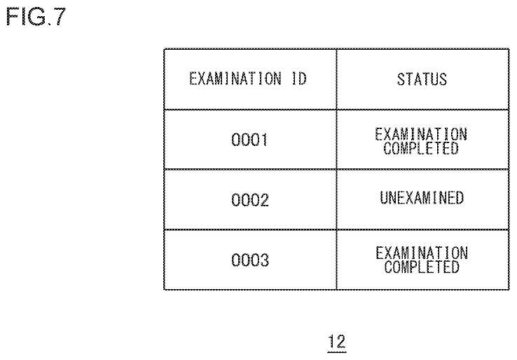

When the acquisition of the image data group by the image data acquisition unit 15 is completed, the status management unit 12 changes the status of the examination order from “unexamined” to “examination completed”. FIG. 7 shows recorded contents of the status management table after the change. The status management unit 12 changes the status of the examination order with the examination ID “0003” from “unexamined” to “examination completed” and then registers the status. When the image data group is transferred from the receiver 4c and the status of the examination order is changed, the provision unit 14 provides information regarding examination orders to which the “unexamined” status is set to all receivers 4 that are detected to be connected. Since the three receivers 4a, 4b, and 4c are connected in this case, the provision unit 14 provides information regarding an examination order with the examination ID “0002” to which the “unexamined” status is set to the receivers 4a, 4b, and 4c. Information regarding examination orders provided in the past is registered in the receivers 4a, 4b, and 4c. However, this information is overwritten by the newly provided information regarding the examination order. Therefore, the information regarding the examination order with the examination ID “0002” is registered in the receivers 4a, 4b, and 4c.

As described above, the provision unit 14 provides information regarding the same examination orders at a predetermined time to all of a plurality of provision units 14 detected to be connected. The information may be provided at multiple points of time. As described above, one such point of time is when the contents of the status management table are updated, and another such point of time is when it is confirmed that a new receiver 4 is connected and there is no need to transfer image data. The provision unit 14 may provide information regarding examination orders to which the “unexamined” status is set to all receivers 4 that are connected at the time when the user logs in to the examination management apparatus 10, at the time when a predetermined operation is performed, at the time when an application is started, and the like.

Described above is an explanation on the present disclosure made based on the embodiments. These embodiments are intended to be illustrative only, and it will be obvious to those skilled in the art that various modifications to constituting elements and processes could be developed and that such modifications are also within the scope of the present disclosure.

What is claimed is:

1. An endoscope system comprising:
a receiver capable of recording an image data group captured by an endoscope;
a cradle comprising a connector that can be connected to the receiver; and
a controller comprising a memory configured to memorize a plurality of pieces of subject information and a processor comprising hardware, wherein
the processor is configured to:
detect that the receiver is connected to the connector; and
provide the plurality of pieces of subject information to the receiver connected to the connector.

2. The endoscope system according to claim 1, comprising a plurality of connectors, wherein
the processor is configured to:
provide a plurality of the pieces of subject information to a plurality of the receivers connected to a plurality of the connectors.

3. The endoscope system according to claim 2, wherein:
the processor is configured to:
provide a plurality of the pieces of subject information that are the same to a plurality of the receivers.

4. The endoscope system according to claim 1, wherein
the processor is configured to:
manage statuses of examination orders; and
provide the receiver with the subject information included in the examination order to which an unexamined status is set.

5. The endoscope system according to claim 4, wherein:
the processor is configured to:
acquire an image data group recorded by the receiver; and
change the status of the examination order from an unexamined status to an examination completed status when the acquisition of the image data group is completed.

6. The endoscope system according to claim 5, wherein
the processor is configured to:
after changing the status of the examination order from the unexamined status to the examination completed status, provide the receiver with the subject information included in another examination order to which the unexamined status is set.

7. The endoscope system according to claim 1, comprising a plurality of the connectors, wherein
the processor is configured to:
when a receiver different from the receiver connected to the connector becomes connected to another connector and the different receiver is not recording an image data group, provide a plurality of the pieces of subject information to a plurality of the connected receivers.

8. An information provision method, comprising:
detecting that a receiver capable of recording an image data group captured by an endoscope is connected to a connector; and
providing a plurality of pieces of subject information to the receiver connected to the connector.

9. The information provision method according to claim 8, wherein
the providing of the subject information includes providing a plurality of the pieces of subject information to a plurality of the receivers connected to a plurality of the connectors.

10. The information provision method according to claim 9, wherein
providing the subject information includes providing a plurality of the pieces of subject information that are the same to a plurality of the receivers.

11. The information provision method according to claim 8, comprising:
managing statuses of examination orders; and
the providing of the subject information includes providing the receiver with the subject information included in the examination order to which an unexamined status is set.

12. The information provision method according to claim 11, comprising:
acquiring an image data group recorded by the receiver, wherein
the managing of the statuses includes changing the status of the examination order from an unexamined status to an examination completed status when the acquisition of the image data group is completed.

13. The information provision method according to claim 12, comprising:
after changing the status of the examination order from the unexamined status to the examination completed status, providing the receiver with the subject information included in another examination order to which the unexamined status is set.

14. The information provision method according to claim 8, comprising:
when a receiver different from the receiver connected to the connector becomes connected to another connector and the different receiver is not recording an image data group, providing a plurality of the pieces of subject information to a plurality of the connected receivers.

15. A receiver comprising:
a recording medium capable of recording an image data group captured by an endoscope; and
a memory configured to memorize a plurality of pieces of subject information provided from an examination management apparatus after a data terminal of the receiver becomes connected to a connector that is connected to the examination management apparatus.

16. The receiver according to claim 15, wherein
the memory is configured to memorize the subject information included in an examination order to which an unexamined status is set.

17. The receiver according to claim 15, wherein
when an image data group is recorded in the recording medium, the receiver transmits the image data group to the examination management apparatus when the data terminal becomes connected to the connector.

18. The receiver according to claim 15, further comprising:
a display that displays the plurality of pieces of subject information.

19. The receiver according to claim 18, wherein
the display displays the plurality of pieces of subject information such that a user can select the plurality of pieces of subject information.
